(19) United States
(12) Patent Application Publication
(10) Pub. No.: US 2023/0044280 A1
(43) Pub. Date: Feb. 9, 2023

(54) ACCESSORY DEVICE FOR AN ENDOSCOPIC DEVICE

(71) Applicant: GI Scientific, LLC, Arlington, VA (US)

(72) Inventor: Scott Miller, Arlington, VA (US)

(21) Appl. No.: 17/936,882

(22) Filed: Sep. 30, 2022

Related U.S. Application Data
(63) Continuation-in-part of application No. PCT/US2021/025272, filed on Mar. 31, 2021.
(60) Provisional application No. 63/003,656, filed on Apr. 1, 2020.
(60) Provisional application No. 63/137,698, filed on Jan. 14, 2021.

(51) Int. Cl. A61B 1/00; A61B 1/01; A61B 1/015; A61B 1/06

(52) U.S. Cl. CPC A61B 1/00137; A61B 1/00096; A61B 1/00089; A61B 1/00098; A61B 1/00165; A61B 1/01; A61B 1/015; A61B 1/0676

(57) ABSTRACT

A support device for an endoscope comprises a tubular member configured for removable attachment to an outer surface of the endoscope near, or at, its distal end and a plurality of projecting elements extending outward from the outer surface of the tubular member and circumferentially spaced from each other. The device includes an optically transparent cover coupled to the tubular member and configured for covering the distal end of the endoscope when the tubular member is attached to the outer surface of the endoscope. The projecting elements provide support for the endoscope, improve visualization and center the scope as it passes through a body lumen, such as the colon. In addition, the cover seals the distal end of the endoscope to protect the scope and its components from debris, fluid, pathogens and other biomatter.

### CROSS-REFERENCE TO RELATED APPLICATIONS

This application is a continuation-in-part (CIP) of International Application No. PCT US/2021/025272, filed Mar. 31, 2021, which claims the benefit of U.S. Provisional Application Nos. 63/003,656, filed Apr. 1, 2020 and 63/137,698, filed Jan. 14, 2021, the entire disclosures of which are incorporated herein by reference for all purposes as if copied and pasted herein.

### FIELD

The present disclosure relates to accessory devices for endoscopic devices, such as endoscopes and more particularly to support devices designed for removal attachment to the working end of endoscopes.

### BACKGROUND

Recent advances in optical imaging technology have allowed many medical procedures to be performed today in a minimally invasive manner. The evolution of the more sophisticated, flexible scope with advanced visual capabilities has allowed access to regions deep within the human body that could only be achieved before with invasive surgical intervention. This modern day convenience has resulted in an increase in the demand for, as well as the number of, endoscopic, laparoscopic, arthroscopic, ophthalmoscopic, or other remote imaging visualization procedures performed every year in the U. S and globally. While these procedures are relatively safe, they are not without risks.

Endoscopy, for instance, is a procedure in which a lighted visualization device called an endoscope is inserted into the patient's body to look inside a body cavity, lumen, organ or in combination, for the purpose of examination, diagnosis or treatment. The endoscope may be inserted through a small incision or through a natural opening of the patient. In a bronchoscopy, the endoscope is inserted through the mouth, while in a sigmoidoscopy or colonoscopy, the endoscope is inserted through the rectum. Unlike most other medical imaging devices, endoscopes are inserted directly into the organ, body cavity or lumen.

In certain endoscopic procedures, for example, flexible instruments designed to view the gastro-intestinal tract are inserted along a body cavity to an internal part, such as the stomach, duodenum, small intestine or large intestine. The instruments are provided with fiberoptic or charge-couple device (CCD) cameras which enable images to be transmitted around bends and images to be produced to displays on a television screen. Accordingly, it is possible to view the inside surfaces of the esophagus, stomach and duodenum using a gastroscope, the small intestine with an enteroscope, part of the colon using a flexible sigmoidoscope and the whole of the large intestine (the bowel) with a colonoscope.

During a colonoscopy, a long flexible tube (e.g., a colonoscope) is inserted into the rectum and advanced through the colon (referred to as “intubation”). When intubation has reached its end point, the colonoscope is then withdrawn back through the colon as the endoscopist examines the surface of the mucosa for disorders, such as polyps, adenomas and the like. While colonoscopic examinations are the most effective techniques to assess the state of health of the bowel, they are inconvenient, uncomfortable, expensive procedures that are time consuming for patients and medical personnel alike. For example, the ascending and descending colon are supported by peritoneal folds called mesentery. As the tip of the endoscope passes along the lumen of the colon, these folds hamper the endoscopist's ability to visualize the entire surface of the mucosa and in particular, detect pre-malignant and malignant lesions tucked away on the proximal face of these folds during extubation.

In addition, the position of the tip of the endoscope may be difficult to maintain from the moment at which a lesion or polyp is detected to the completion of any therapeutic procedure. As the colonoscope is withdrawn, the tip does not travel back at a constant speed but rather with jerks and slippages, particularly when traversing a bend or length of colon where the bowel has been collapsed over the endoscope shaft during intubation. The tip of the device may, at any moment, slip backwards thereby causing the clinician to lose position. If tip position is lost, the clinician is required to relocate the lesion or polyp for the therapeutic procedure to be continued.

Another challenge with these procedures is that the bowel is long and convoluted. In certain locations, it is tethered by peritoneal bands and in others it lies relatively free. When the tip of the endoscope encounters a tight bend, the free part of the colon loops as more of the endoscope is introduced, making it difficult for the operator to negotiate around the bend. This leads to stretching of the mesentery of the loop (the tissue that carries the nerves and blood vessels to the bowel). If the stretching is continued or severe while the endoscopist pushes round the bend, the patient may experience pain or a reduction in blood pressure.

Attempts have been made to try to overcome the problems associated with colonoscopic procedures. Endoscope support devices, or “cuffs”, have been developed that include a tubular member that grips the outer surface of the distal end of the scope and a plurality of spaced projecting elements extending outward from the tubular member. The projecting elements are flexible and designed to fan or spread out to provide support for and dilate a lumen wall of a body passage into which the medical scoping device has been inserted. The projecting elements are designed to elongate and smooth out the folds of the intestine as the endoscope is withdrawn therethrough.

While these new support devices have overcome some of the challenges of colonoscopic procedures, they still suffer from a number of drawbacks. For example, since the projecting members are spaced around the circumference of the tubular member, they do not contact the entire circumference of the intestine. In particular, the projecting members have gaps therebetween where no contact is made.

Another challenge with existing support devices is that they are typically attached to the outer surface of the endoscope so that they do not block or otherwise obstruct the camera lens at the distal tip of the scope. Therefore, they do not seal the scope from the surrounding environment.

Endoscopes are typically reused, which means that, after an endoscopy, the endoscope goes through a cleaning, disinfecting or sterilizing, and reprocessing procedure to be introduced back into the field for use in another endoscopy on another patient. In some cases, the endoscope is reused several times a day on several different patients.

While the cleaning, disinfecting and reprocessing procedure is a rigorous one, there is no guarantee that the endoscopes will be absolutely free and clear of any form of contamination. Modern day endoscopes have sophisticated and complex optical visualization components inside very small and flexible tubular bodies, features that enable these scopes to be as effective as they are in diagnosing or treating patients. However, the tradeoff for these amenities is that they are difficult to clean because of their small size, and numerous components. These scopes are introduced deep into areas of the body which expose the surfaces of these scopes to elements that could become trapped within the scope or adhere to the surface, such as body fluids, blood, and even tissue, increasing the risk of infection with each repeated use.

Endoscopes used in the gastrointestinal tract have an added complexity in that they are in a bacteria rich environment. This provides an opportunity for bacteria to colonize and become drug resistant, creating the risk of significant illness and even death for a patient. Moreover, in addition to the health risks posed by bacterial contamination, the accumulation of fluid, debris, bacteria, particulates, and other unwanted matter in these hard to clean areas of the scope also impact performance, shortening the useful life of these reusable scopes.

Accordingly, it is desirable to provide accessory devices that reduce the risk of contamination and infection, while also improving the performance of endoscopic devices. It is particularly desirable to provide a support device for an endoscope that allows the user to fully support and dilate the luminal wall of a body passage to improve visualization of the luminal wall, while also protecting the working end of the scope from bacterial or other microbial contamination.

### SUMMARY

The present disclosure provides accessories, such as support devices, for endoscopic devices, such as endoscopes. The support devices provide support for the endoscope, center the scope as it passes through a body lumen, such as the colon, and improve visualization of the luminal walls. In addition, the support devices seal the distal end of the endoscope to protect the scope and its components from debris, fluid, pathogens and other biomatter.

In one aspect, a support device for an endoscope comprises a tubular member configured for removable attachment to an outer surface of the endoscope near, or at, its distal end and a plurality of projecting elements extending outward from the outer surface of the tubular member and circumferentially spaced from each other. The device includes an optically transparent cover coupled to the tubular member and configured for covering the distal end of the endoscope when the tubular member is attached to the outer surface of the endoscope. The cover and the tubular member create a seal over the distal end of the endoscope, thereby protecting the scope and its components and reducing the risk of debris, fluid and other matter accessing hard-to-clean areas of the endoscope, potentially causing infection risk.

The cover may be substantially aligned with the light transmitter and/or camera lens of the scope to allow for viewing of the surgical site through the support device. The cover may include one or more openings that allow an instrument to pass through the support device from a working or biopsy channel of the endoscope to the surgical site. The openings may be sealable to prevent or minimize air, fluid or other foreign matter from passing through the openings and into the support device. The tubular member or the cover may include one or more hollow instrument channels extending from the openings of the cover to the working end of the endoscope.

In certain embodiments, the cover is spaced from the distal end of the endoscope when the tubular member is attached to the outer surface of the endoscope. The cover is preferably spaced from the lens of the endoscope by a length less than a minimum focal distance of the scope which generally depends on the type of lens. This ensures that the cover does not interfere with the view provided by the camera lens.

The cover may be integral with the tubular member to form a single unitary body that attaches to the distal end of the endoscope. Alternatively, the cover may be removably coupled to the tubular member.

The tubular member may have an inner surface configured for gripping the outer surface of the endoscope to hold the support device in place during movement of the endoscope through, for example, a body passage. Alternatively, the support device may include an attachment member for removably support the tubular member to the scope.

The projecting elements may each comprise a base coupled to the tubular member and a substantially flexible arm extending from the base. The flexible arm of each projecting element is preferably movable between a first position, wherein the flexible arm generally flattens out against the tubular member to facilitate advancement of the endoscope through a body lumen, to a second position, wherein the flexible arms extend laterally outward from the tubular member. In certain embodiments, the flexible arms are substantially perpendicular to a longitudinal axis of the tubular member in the first position to allow the endoscope to be advanced through a body lumen without being hindered by the projecting elements. In certain embodiments, the flexible arms extend substantially perpendicular to the longitudinal axis of the tubular member in the second position and may be movable to change angles as they encounter folds or other interruptions in the luminal wall as the endoscope is withdrawn through the lumen.

The projecting elements provide support for the endoscope by fanning out to contact the folds in the wall of the body lumen. The projecting elements may comprise a resiliently deformable material capable of elongating, flattening and/or everting these folds. In addition, the projecting elements dilate the body lumen and improve visualization of the tissue on either side of the folds. The projecting elements also help to center the scope, minimize “looping” of the colonic wall and inhibit loss of tip position, thereby reducing the overall time of the procedure and minimizing complications.

The projecting elements may comprise any suitable shape, such as cylindrical, conical, tapered, rectangular and the like and may form be in the form of cones, wedges, paddles, spines, fins, bristles, spikes or the like. The projecting elements may be formed integrally with the outer surface of the tubular member or they may be attached thereto.

The bases of the projecting elements may be raised so that they form a bump or bulge on the outer surface of the tubular member. The projecting elements may be hinged or movable about their bases. Alternatively, they may comprise a suitable biocompatible material that is flexible and resiliently deformable so that the projecting members bend relative to the bases. The material may have a stiffness that allows the projecting elements to deform slightly when contacting the colonic wall so that the tip of the projecting elements bends out rather than pressing into or impinging onto the colonic wall causing trauma.

In certain embodiments, the support device includes one or more sensors on the tubular member and/or the cover for detecting one or more physiological parameters of the patient. The physiological parameters may include a temperature of the tissue, a type of fluid in, or around, the tissue, pathogens in, or around, the tissue, a dimension of the tissue, a depth of the tissue, a tissue disorder, such as a lesion, tumor, ulcer, polyp or other abnormality, biological receptors in, or around, the tissue, tissue biomarkers, tissue bioimpedance, a PH of fluid in, or around the tissue or the like.

The support device may be coupled to a processor that includes one or more software applications with one or more sets of instructions to cause the processor to recognize the images captured by the imaging device and/or the physiological parameters detected by the sensors and to determine if the tissue contains a medical condition. In certain embodiments, the software application(s) are configured to compare the tissue images with data related to one or more medical disorders, images of certain medical disorders or other data related to such disorders, such as tissue color, texture, topography and the like. In an exemplary embodiment, the software application(s) or processor may include an artificial neural network (i.e., an artificial intelligence or machine learning application) that allows the processor to develop computer-exercisable rules based on the tissue images captured from the patient and the data related to certain medical disorders to thereby further refine the process of recognizing and/or diagnosing the medical disorder.

The system may further include a memory in the processor or another device coupled to the processor. In one such embodiment, the memory further contains images of representative tissue, and the processor is configured to compare the current images captured by the endoscope with the representative tissue. The memory may, for example, contain images of tissue from previous procedures on the same patient. In this embodiment, the processor is configured to compare the images taken during the current procedure with images from previous procedures. In some cases, these previous images include a topographic representation of an area of the patient, such as the GI tract or other selected area. The processor is further configured to determine, for example, if the physician has examined the entire area selected for examination (e.g., by comparing the current images with previous images that represent the entire area). The processor may make this determination in real-time to alert the physician that, for example, the examination has not been completed. In other embodiments, the processor may be configured to save the images so that the physician can confirm that the examination has been complete.

In other embodiments, the previous images may include selected tissue or areas from the patient, such as a medical disorder. The medical disorder may, for example, include a tumor, polyp, ulcer, inflammation, abnormal or diseased tissue or other disorder. In this embodiment, the processor comprises one or more software applications with sets of instructions that allow the processor to compare the current images of the disorder with previous images to, for example, determine if the disorder has changed between the procedures. For example, the software applications may have a set of instructions that compare previous and current images of cancerous tissue and then determine if the cancerous tissue has grown or changed in any material aspect. In another example, the processor may determine if a previously-removed polyp or tumor has returned or was completely removed in a previous procedure.

In other embodiments, the memory contains images of representative tissue from patients other than the current patient. For example, the representative tissue may comprise a series of images of certain types of disorders, such as a tumor, polyp, ulcer, inflammation or a diseased tissue. In this embodiment, the system further includes one or more software applications coupled to the processor and configured to characterize the disorder in the patient based on the images captured by the endoscope and the images of the representative tissue. The software applications may include an artificial neural network (e.g., an artificial intelligence or machine-learning program) that includes a set of instructions that allows the software applications to “learn” from previous images and apply this learning to the images captured from the patient. The software application can be used to, for example, supplement the physician's diagnosis of the disorder based on the series of images of other similar disorders and/or to reduce the variation in diagnostic accuracy among medical practitioners.

In certain embodiments, the software application may be configured to analyze images from the entire area of the procedure and compare these images with data or other images in the memory. The software application may be further configured to detect a potential disorder in the selected area of examination based on the images and data within memory. Detection of a potential disease or disorder by the software application during the endoscopic diagnosis makes it possible to prevent a detection target from being overlooked by a medical practitioner, thereby increasing the confidence of an endoscopic diagnosis.

In certain embodiments, the memory includes a variety of different patient characteristics that create a patient profile, such as age, ethnicity, nationality, race, height, weight, baseline vitals, such as blood pressure, heart rate and the like, hematology results, blood chemistry or urinalysis results, physical examinations, medication usage, blood type, BMI index, prior medical history (e.g., diabetes, prior cancerous events, irritable bowel syndrome or other GI tract issues, frequency of colonoscopies, frequency and growth rate of polyps, etc.) and other relevant variables. The memory may be linked to a central repository in a computer network or similar type network that provides similar profiles from a multitude of different patients in different locations around the country. In this manner, an individual health care practitioner or hospital staff can access hundreds or thousands of different patient profiles from various locations around the country or the world.

In this embodiment, the processor may include an artificial neural network capable of classifying the patient based on a comparison of his/her individual profile and the other profiles in the network. This classification may include a relevant risk profile for the patient to develop certain disorders or diseases. Alternatively, it may allow the software application(s) to recognize the medical disorder based on the images and/or data collected during the procedure.

The system may be configured to capture data relevant to the actual size and depth of tissue, lesions, ulcers, polyps, tumors and/or other abnormalities within the patient. For example, the size of a lesion or ulcer may range from a scale of 100 micrometers to a few centimeters. The software applications may include sets of instructions to cause the processor to collect this depth information and to classify the depth as being superficial, submucosal, and/or muscularis. The processor also be configured to capture data regarding the prevalence of impact of lesions or ulcers within a specific region of the patient.

Data gathered from any of the sources above may be used to train an algorithm, such as an AI algorithm, to predict exacerbations or flare-ups. Information input regarding medication may be used to, for example, predict or otherwise consider a patient's response to medication and enable a health care provider, patient, caregiver or other party to tailor medication treatments. Data from different sources described above may be combined in various permutations in order to enable predictive diagnostics and/or treatment recommendations.

In another aspect, the system may further include one or more sensors on, or within, an outer surface of the tubular member, the projecting elements and/or optically transparent cover. The sensors are configured to detect a physiological parameter of tissue around the support device. The physiological parameter may include, for example, a temperature of the tissue. a dimension of the tissue, a depth of the tissue, tissue topography, tissue biomarkers, tissue bioimpedance, temperature, PH, histological parameters or another parameter that may be used for diagnosing a medical condition.

The system further includes a connector configured to couple the sensor to a processor. The processor may also receive images from the camera on the endoscope. In certain embodiments, the processor is configured to create a topographic representation of the tissue based on the images and/or the physiological parameter(s). In this embodiment, the system may further comprise a memory containing data regarding the physiological parameter from either the current patient or a plurality of other patients. The system includes a software application coupled to the processor and configured to diagnose the patient based on the physiological parameter detected by the sensor and the images captured by the endoscope. The software application may include an artificial neural network (e.g., an artificial intelligence or machine-learning program) that allows the software application to “learn” from previous physiological parameters of the patient, or from physiological parameters of other patients and then apply this learning to the data captured from the patient. The system may include, for example, a trained machine learning algorithm configured to develop from the images of representative tissue at least one set of computer-executable rules useable to recognize a medical condition in the tissue images captured by the endoscope. For example, the software application may be configured to diagnose one or more disease parameters based on the physiological parameter and/or the images.

In another aspect, a support device for an endoscope comprises a tubular member configured for removable attachment to an outer surface of the endoscope near, or at, the distal end. The support device further includes first and second rings of projecting elements extending outward from the outer surface of the tubular member. The projecting elements within the first and second rings are spaced from each other around a circumference of the tubular member to define gaps therebetween. The first ring is spaced longitudinally from the second ring and the projecting elements of the second ring are aligned longitudinally with the gaps between the projecting elements in the first ring. The projecting elements of the first ring may also be aligned longitudinally with the gaps between the projecting elements in the second ring.

The projecting elements in the first and second ring intermesh with each other to provide a more consistent and uniform contact surface between the tips of the projecting elements and the colonic wall. This allows the projecting elements to elongate, flatten and/or evert folds in the colonic wall more uniformly, especially around curves and in complex anatomy. They also aid in navigating around curves in the colon, inhibit or completely prevent looping of the endoscope and provide a more consistent centering of the endoscope as it passes through the colon.

In certain embodiments, the first and second rings may be spaced from each other in the longitudinal direction by a distance of at least about 2.5 cm, preferably about 2.5 cm to about 4.0 cm, or about 2.6 cm to about 3.0 cm.

In another aspect, a support device for an endoscope comprises a tubular member configured for removable attachment to an outer surface of the endoscope near, or at, the distal end and a plurality of projecting elements extending outward from the outer surface of the tubular member. The projecting elements are spaced from each other around a circumference of the tubular member. The projecting elements are also spaced from a distal end of the tubular member by a distance of greater than about 20 mm.

In certain embodiments, the support device includes a plurality of rings of the projecting elements extending outward from the outer surface of the tubular member. Each of the rings are spaced from each other in the longitudinal direction. The distalmost ring or the ring closest to the distal end of the tubular member is spaced from the distal end of the tubular member by a distance of greater than about 20 mm.

In another aspect, a method for visualizing a surface within a patient comprises attaching a tubular member of a support device to a distal end of an endoscope and sealing the distal end of the scope with an optically transparent cover. The endoscope is advanced through a body lumen, such as the colon, and then retracted back through the body lumen to allow an operator to view an inside surface of the lumen. At least a portion of the inner surface of the body lumen is dilated with one or more projecting elements extending from an outer surface of the tubular member. The projecting elements elongate and smooth out the folds of the intestine as the endoscope is withdrawn therethrough.

In embodiments, the cover is substantially aligned with the light transmitter and/or camera lens of the scope to allow for viewing of the surgical site through the support device. The cover may be spaced from the distal end of the endoscope when the tubular member is attached to the outer surface of the endoscope. The cover is preferably spaced from the lens of the endoscope by a length less than a minimum focal distance of the scope to ensure that the cover does not interfere with the view provided by the camera lens.

In embodiments, the support device may be centered within the body lumen with the projecting elements. The support device may be further provided with first and second rings of projecting elements the intermesh with each other to provide a more consistent and uniform contact surface between the tips of the projecting elements and the colonic wall. This allows the projecting elements to elongate folds in the colonic wall more uniformly, especially around curves and in complex anatomy.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a perspective view of a support device attached to a distal end of an endoscope;

FIG. 2 is a side view of the support device of FIG. 1;

FIG. 3 is a front view of the support device of FIG. 1;

FIG. 4A is a schematic illustration of an endoscope and the support device of FIG. 1 during advancement through the colon of a patient;

FIG. 4B is a schematic illustration of the support device and endoscope, during withdrawal back through the colon towards the anus of the patient;

FIG. 5 is a schematic view of a system for monitoring, mapping, diagnosing, treating and/or evaluating tissue within a patient; and

FIG. 6 a partial cross-sectional view of the proximal portion of a representative endoscope coupled to a representative processor.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

The term “endoscope” in the present disclosure refers generally to any scope used on or in a medical application, which includes a body (human or otherwise) and includes, for example, a laparoscope, duodenoscope, endoscopic ultrasound scope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

While the following description is primarily directed to support devices for endoscopes, it should be understood that the devices disclosed herein may be used as an accessory to other endoscopic devices configured for advancement or withdrawal through an opening of a patient and through a body lumen, such as catheters and endoscopic instruments. For purposes of this disclosure, an opening means natural orifice openings through any pre-existing, natural opening into the patient, such as the mouth, sinus, ear, urethra, vagina or anus, or any access port provided through a patient's skin into a body cavity, internal lumen (i.e., blood vessel), etc. or through incisions, and port-based openings in the patient's skin, cavity, skull, joint, or other medically indicated points of entry. The endoscopic device may also be configured to pass through a working or biopsy channel within an endoscope (i.e., through the same access port as the endoscope). Alternatively, the endoscopic device may be configured to pass through an opening that is separate from the endoscope access point.

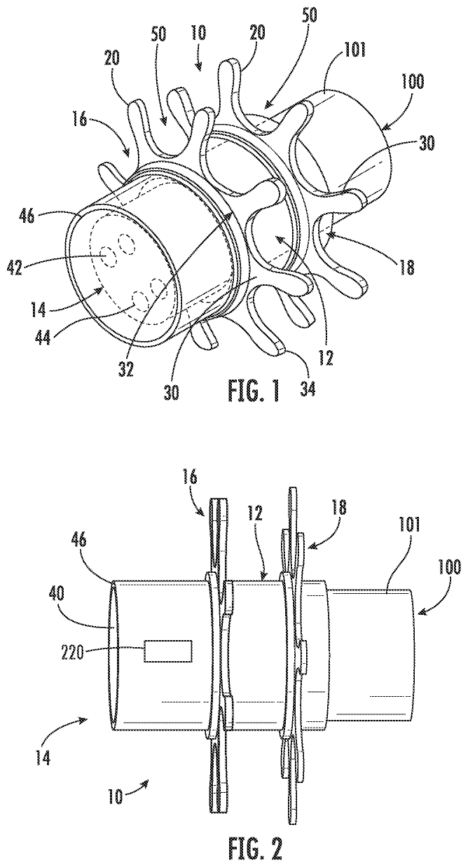

Referring now to FIGS. 1-3, a support device 10 comprises a tubular member 12, a cover 14 and first and second rings 16, 18 of projecting elements 20 extending from an outer surface of tubular member 12. Tubular member 12 includes an inner surface (not shown) at least part of which grips the distal portion of the shaft 101 of a medical device, such as an endoscope 100. Tubular member 12 holds support device 10 in place relative to shaft 101 as the medical device is inserted into the patient and, for example, advanced or withdrawn through a body lumen, such as the colon or other passage in the GI tract of a patient.

In one version of the support device 10, the support device 10 is molded from a material selected from silicone gels, silicone elastomers, epoxies, polyurethanes, and mixtures thereof. The silicone gels can be lightly cross-linked polysiloxane (e.g., polydimethylsiloxane) fluids, where the cross-link is introduced through a multifunctional silane. The silicone elastomers can be cross-linked fluids whose three-dimensional structure is much more intricate than a gel as there is very little free fluid in the matrix. In another version of the support device 10, the material is selected from hydro gels such as polyvinyl alcohol, poly(hydroxyethyl methacrylate), polyethylene glycol, poly(methacrylic acid), and mixtures thereof. The material for the optical support 10 may also be selected from albumin based gels, mineral oil based gels, polyisoprene, or polybutadiene. Preferably, the material is viscoelastic.

Tubular member 12 may be formed from a variety of materials. Tubular member 12 can be a semi-solid gel, which is transparent and flexible, that attaches to a wide variety of endoscopes. In certain embodiments, tubular member 12 comprises an elastic material that can be stretched sufficiently to extend around the distal or working end of shaft 101. Tubular member 12 also comprises a resilient material that compresses against shaft 101 to hold support device 10 in place. Alternatively, support device 10 may include a separate attachment element, such as a clamp, brace, clip and the like for removably mounting tubular member 12 to shaft 101.

Cover 14 comprises at least an optically transparent distal surface 40 and has a shape configured to align with and cover a light transmitter 42 and lens 44 at the distal end of endoscope 100. Cover 14 may be formed integrally with tubular member 12, or it may be a separate component that is attached or molded thereto. In some embodiments, cover 14 is a substantially disc-shaped component attached to, or integrally formed with, a circumferential distal end 46 of tubular member 12. In other embodiments, cover 14 may comprise a substantially cylindrical component that is hollow inside and has a proximal circumferential surface that is attached to, or integrally formed with, the circumferential distal end 46 of tubular member 12.

The distal surface 40 of cover 14 may be generally flat, or it may have a slightly curved surface to facilitating clearing of the field of view by pushing any fluid or matter from the center of distal surface 40 to its boundaries.

In any of these embodiments, cover 14 and tubular member 12 are designed to seal the working end of the endoscope 100 when tubular member 12 is attached to shaft 101 to protect the scope and its components, particularly the camera lens 44. This reduces the risk of debris, fluid and other matter ending up in the camera lens 44, and other hard-to-clean areas potentially causing infection risk.

In certain embodiments, cover 14 is spaced from the camera lens 44 of scope 100 when tubular member 12 is attached to shaft 101. Cover 14 is preferably spaced from lens 44 by a length less than a minimum focal distance of the endoscope to ensure that cover 14 does not interfere with the field of view provided by the lens 44.

In one example configuration, the endoscope 100 may be a fixed-focus endoscope having a specific depth of field. In this example, distal surface 40 may be spaced apart from lens 44 of the endoscope 100 by a length D equal to a reference distance selected from values in the depth of field distance range of the endoscope 100. In one example configuration, the endoscope 100 may have a depth of field in the range of 2 to 100 millimeters. In this case, distal surface 40 is spaced apart from lens 44 by a length in the range 2 to 100 millimeters. Preferably, the length D equals a reference distance that is in the lower 25% of values in the depth of field distance range of the endoscope 100. In one example configuration, the endoscope 100 may have a depth of field in the range of 2 to 100 millimeters. In this case, the length D equals a value of 2-26 millimeters. More preferably, the length D equals a reference distance that is in the lower 10% of values in the depth of field distance range of the endoscope 100. In one example configuration, the endoscope 100 may have a depth of field in the range of 2 to 100 millimeters. In this case, the length D equals a value of 2-13 millimeters. Most preferably, the length D equals a reference distance that is greater than or equal to the lowest value (e.g., 2 millimeters) in the depth of field distance range of the endoscope 100. In one version of the support 10, the length D is 7-10 millimeters, or a typical distance that the endoscope 100 is held from tissue that would be receiving an endoscopic treatment or therapy.

The design of the length D for the support device 10 should also take into consideration the characteristics of the materials that compose the support device 10, such as any possible compression of the support 10 when it is held against a surface. For example, if the support device 10 may be compressed 1 millimeter when held against a surface and the lowest value in the depth of field distance range of the endoscope 100 is 2 millimeters, then the length D should be greater than or equal to 3 millimeters to compensate for this possible compression.

Rings 16, 18 are longitudinally spaced from each other and from the distal end of cover 14. In certain embodiments, the distalmost ring 16 is spaced from the distal surface 40 of cover 14 by at least about 20 mm, preferably between about 20 mm and about 40 mm, more preferably between about 25 mm and about 30 mm

Rings 16, 18 are preferably spaced from each other by a distance of at least about 2.5 cm, preferably between about 2.5 cm to about 4 cm, or between about 3.0 cm and 3.5 cm Thus, the proximal most ring 18 is preferably spaced at least about 4.5 cm, preferably between about 5.0 cm to about 6.0 cm from the distal surface of cover 14. Support device 10 may include more than two rings, between 2 and 50 rings, or between about 2 and 20 rings. Each ring 16, 18 may comprise 4 to 16 projecting elements 20, or more preferably between about 5 to 10 projecting elements 20.

Projecting elements 20 may be in the form of bristles, spikes, spines, fins, wedges, paddles, cones or the like and/or may have cylindrical, conical, tapered, rectangular or other shapes. Projecting elements 20 may have substantially flat surfaces or they may be curved. For example, the surfaces of projecting elements 20 that face the longitudinal direction may be flat or curved. Similarly, the surfaces of each projecting element facing in the lateral direction may be flat or curved.

Projecting elements 20 each include a base 30 and a tip 34, that may either be rounded or blunted. Base 30 is attached to a circumferential ring 32 that extends around tubular member 12. Projecting elements 20 and ring 32 may be molded together as a single unitary component, or they may be molded separately and coupled to each other in any suitable fashion. Similarly, circumferential ring 32 may be formed integrally with the outer surface of tubular member 12 or attached or molded thereto.

Projecting elements 20 may have one or more openings between the base 30 and the tip 34. These openings may extend partially or fully through projecting elements 20, and may have a number of shapes, such as triangular, conical, rectangular, square or the like.

Projecting elements 20 provide support for the endoscope by fanning out to contact the folds in the wall of a body lumen. Projecting elements 20 may comprise a resiliently deformable material capable of elongating, flattening and/or everting these folds. In addition, projecting elements 20 dilate the body lumen and improve visualization of the tissue on either side of the folds. Projecting elements 20 also help to center the scope, minimize “looping” of the colonic wall and inhibit loss of tip position, thereby reducing the overall time of the procedure and minimizing complications.

Projecting elements 20 define gaps 50 therebetween. Gaps 50 generally form a U-shaped opening or cavity between each of the projecting elements 20, although the specific shape of these openings will vary depending on the shape of each of the projecting elements 20. For example, projecting elements 20 may have a substantially rectangular shape in which case gaps 50 will have a substantially rectangular shape. Alternatively, projecting elements 20 may have a conical shape such that gaps 50 have straighter edges, that are V-shaped, U-shaped or a combination of the two.

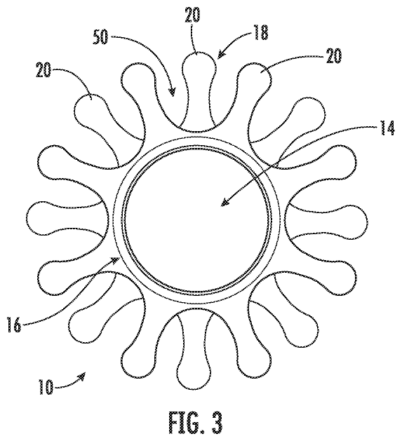

In one embodiment, projecting elements 20 in ring 16 are aligned longitudinally with gaps 50 in ring 18. Likewise, projecting elements 20 in ring 18 are aligned longitudinally with gaps 50 in ring 16. Thus, the projecting elements 20 in adjacent rings are offset from each other such that they “cover” the gaps between the projecting elements (see FIG. 3). Projecting elements 20 may be located in the center of gaps 50, or they may be located slightly off-center of the gaps 50. Projecting elements 20 may be sized to “cover” substantially the entire circumferential distance of the gaps 50, or they may only cover a portion of the gaps 50.

This design allows the projecting elements to elongate folds in the colonic wall more uniformly, especially around curves and in complex anatomy. The intermeshed projecting elements 20 also aid in navigating around curves in the colon, inhibit or completely prevent looping of the endoscope and provide a more consistent centering of the endoscope as it passes through the colon.

In certain embodiments, support device 10 comprises more than 2 rings of projecting elements. For example, support device 10 may include three rings or more. In one such embodiment, the projecting elements 20 in each ring is substantially aligned with the gaps 50 in adjacent rings. In other embodiment, the projecting element 20 in two successive rings are aligned with each other or slightly offset with each other, but both aligned with the gap 50 in the adjacent rings. In this embodiment, two projecting elements (one in each successive ring) “cover” the gaps in adjacent rings.

The projecting elements 20 in each ring may have substantially the same shape or length. Alternatively, the projecting elements 20 in some of the rings may have different shapes or lengths. In certain embodiments, the projecting elements 20 in a single ring may have different shapes or lengths. For example, the projecting elements may alternate around the circumference of tubular member 12 with longer and shorter projecting elements 20.

You can see capillaries changing diameter: AI approach: apply our capability directly on the tissue. Real-time assessment. Thermography, measure tissue condition.

In some embodiments, projecting elements 20 may be rotatably coupled to rings 32 such that elements 20 are hinged and capable of moving relative to rings 32. In other embodiments, elements 20 are made of a flexible, deformable material that allows elements 20 to move relative to rings 32.

In any of these embodiments, projecting elements 20 are capable of moving between a first position, where tips 34 extend towards the proximal end of endoscope 100 to a second position where tips 34 extend at a transverse angle relative to tubular member 12. In certain embodiments, the tips 34 extend substantially parallel to the longitudinal axis of tubular member 12 (and thus endoscope 100) in the first position. The tips 34 may be configured to move into a substantially perpendicular angle to tubular member 12 in the second position (as shown in FIG. 1). In certain embodiments, the tips 34 may even extend to an obtuse angle to tubular member 12 such that they bend forwards towards the distal end of support device 10.

Projecting elements 20 are designed to open out and extend away from tubular member 12 when endoscope 100 is withdrawn through a body lumen of a patient. This creates a fan or spread of projecting elements 20 that gently support the wall of the body passage and especially the colon. When the colon is tortuous, withdrawing the colonoscope draws the colon back, opening up the path ahead. Forward motion simply causes projecting elements 20 to collapse against the outer surface of tubular member 12 so that they are substantially parallel to the longitudinal central axis of the scope, which allows the scope to be advanced without hindrance.

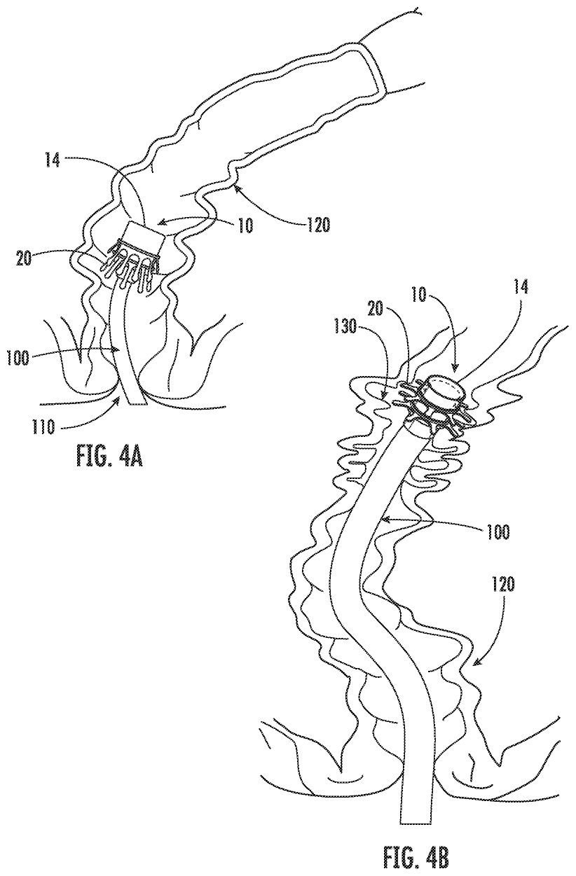

Referring now to FIGS. 4A and 4B, a method of using support device 10 with an endoscope 100 will now be described. The endoscope 100 and support device 10 are inserted via an anus 110 into colon 120 of an individual under investigation, as is well known in the art. On insertion and advancement through the colon 120, projecting elements 20 generally flatten out into a position substantially parallel with the longitudinal axis of endoscope 100, which allows the scope to be advanced without hindrance.

Once the physician reaches a target site in the colon 120, endoscope 100 will be withdrawn back through colon 120 in order to conduct the examination. As the scope is withdrawn, projecting elements 20 fan outward from tubular member 12 to dilate the lumen and flatten the colonic folds 130. This improves visualization and allows the physician to inspect the colon between these folds. In addition, projecting elements 120 assist in centering endoscope 100 as it is advanced and withdrawn through the colon 120.

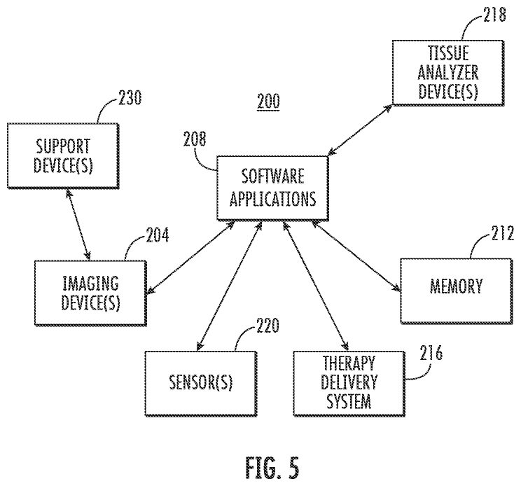

In certain embodiments, support device 10 includes one or more sensors 220 (see also FIG. 5) on tubular member 12 and/or cover 14 for detecting one or more physiological parameters of the patient. The physiological parameters may include a temperature of the tissue, a type of fluid in, or around, the tissue, pathogens in, or around, the tissue, a dimension of the tissue, a depth of the tissue, a tissue disorder, such as a lesion, tumor, ulcer, polyp or other abnormality, biological receptors in, or around, the tissue, tissue biomarkers, tissue bioimpedance, a PH of fluid in, or around the tissue or the like. Suitable sensors for use with the present invention may include PCT and microarray based sensors, optical sensors (e.g., bioluminescence and fluorescence), piezoelectric, potentiometric, amperometric, conductometric, nanosensors or the like.

The system further includes a connector configured to couple the sensor to a processor. The connector may, for example, be a wireless connector (e.g. Bluetooth or the like), or it may be a wired connector that extends through the endoscopic device.

In another aspect, devices, systems, and methods for recognizing, diagnosing, mapping, sensing, monitoring and/or treating selected areas within a patient's body are disclosed. In particular, in at least some aspects, the devices, systems and methods of the present disclosure may be used to diagnose, monitor, treat and/or predict tissue conditions by mapping, detecting and/or quantifying images and physiological parameters in a patient's body, such as size, depth and overall topography of tissue, biomarkers, bioimpedance, temperature, PH, histological parameters, lesions or ulcers, bleeding, stenosis, pathogens, diseased tissue, cancerous or precancerous tissue and the like. The devices, systems, and methods described herein may be used to monitor, recognize and/or diagnose a variety of conditions including, but not limited to, gastrointestinal conditions such as nausea, abdominal pain, vomiting, pancreatic, gallbladder or biliary tract diseases, gastrointestinal bleeding, irritable bowel syndrome (IBS), gallstones or kidney stones, gastritis, gastroesophageal reflux disease (GERD), inflammatory bowel disease (IBD), Barrett's esophagus, Crohn's disease, polyps, cancerous or precancerous tissue or tumors, peptic ulcers, dysphagia, cholecystitis, diverticular disease, colitis, celiac disease, anemia, and the like.

FIG. 5 depicts an exemplary diagnostic, mapping, treating and/or monitoring system 200 for use with device 10 and endoscope 100. Monitoring system 200 may include, among other things, one or more imaging devices 204 that may also include a support device 230 coupled to an imaging device 204 such as one of the support devices 10 described above in FIGS. 1-4. System 200 further includes one or more software applications 208, a memory 212, one or more therapy delivery systems 216, one or more tissue analyzing devices 218 and one or more sensors 220 that may be incorporated into the imaging devices 204 and/or the support devices 230, therapy delivery systems 216 or both. Software applications 208 include one or more algorithms that include sets of instructions to allow a processor to build a model based on the data obtained from the patient by sensors 220, imaging devices 204, support devices 230, tissue analyzing devices 218 and/or certain data stored within memory 212. A more complete description of a suitable processing system for use with the support devices disclosed herein can be found in commonly-assigned PCT Publication No. US 2021/025272, filed Apr. 1, 2021, the complete disclosure of which is incorporated herein by reference in its entirely for all purposes.

In certain embodiments, memory 212 may contain images and/or data captured during a procedure on a patient. Memory 212 may also contain images and/or data of representative tissue, such as images and/or data of tissue from previous procedures on the same patient. In some cases, these previous images include a topographic representation of an area of the patient, such as the GI tract or other selected area. In other embodiments, the previous images may include selected tissue or areas from the patient, such as a medical disorder. In other embodiments, memory 212 contains images and/or data of representative tissue from patients other than the current patient. For example, the representative tissue may comprise a series of images of certain types of disorders, such as a tumor, polyp, ulcer, inflammation or abnormal or diseased tissue. These images may, for example, include hundreds or even thousands of different images of certain types of disorders (e.g., a particular type or grade of cancerous tissue). These images are available for software applications 208 to compare against the images collected by imaging devices 204 and/or support devices 230 to facilitate the recognition of a disorder in the patient, as discussed in more detail below.

Software application(s) 208 include sets of instructions to allow processor 202 to analyze signals from imaging device 204 and/or support device 230 and other inputs, such as sensors 220, medical records, medical personnel, and/or personal data; and extract information from the data obtained by imaging device 204 and the other inputs. Processor 202 or any other suitable component may apply an algorithm with a set of instructions to the signals or data from imaging device 204 and/or support device 230, sensors 220 and other inputs. Processor 202 may store information regarding algorithms, imaging data, physiological parameters of the patient or other data in memory 212. The data from inputs such as imaging device 204 may be stored by processor 202 in memory 212 locally on a specialized device or a general-use device such as a smart phone or computer. Memory 212 may be used for short-term storage of information. For example, memory 212 may be RAM memory. Memory 212 may additionally or alternatively be used for longer-term storage of information. For example, memory 212 may be flash memory or solid state memory. In the alternative, the data from imaging device 204 may be stored remotely in memory 212 by processor 202, for example in a cloud-based computing system.

In certain embodiments, software applications 208 may be aided by an artificial neural network (e.g., machine learning or artificial intelligence). Machine learning is the scientific study of algorithms and statistical models that computer systems use to perform a specific task without using explicit instructions, relying on patterns and inference instead. Machine learning algorithms build a mathematical model based on sample data, known as “training data”, in order to make predictions or decisions without being explicitly programmed to perform the task. The artificial neural network may use algorithms, heuristics, pattern matching, rules, deep learning and/or cognitive computing to approximate conclusions without direct human input. Because the AI network can identify meaningful relationships in raw data, it can be used to support diagnosing, treating and predicting outcomes in many medical situations.

The artificial neural network includes one or more trained machine learning algorithms that process the data received from imaging devices 204, support devices 230 and/or sensors 220 and compares this data with data within memory 212. The artificial neural network may, for example, compare data and/or images collected from other patients on certain disorders and compare this data and/or images with the images collected from the patient. The artificial neural network is capable of recognizing medical conditions, disorders and/or diseases based on this comparison. In another example, the artificial neural network may combine data within memory 212 with images taken from the target site(s) of the patient to create a two or three dimensional map of the topography of a certain area of the patient, such as the gastrointestinal tract. In yet another example, the algorithms may assist physicians with interpretation of the data received from sensors 220, support devices 230 and/or imaging device 104 to diagnose disorders within the patient.

In one embodiment, software application(s) 208 include sets of instructions for the processor 202 to compare the images captured by imaging device 204 with the representative tissue in memory 212. Memory 212 may, for example, contain images and/or data of tissue from previous procedures on the same patient. In this embodiment, software application(s) 208 include sets of instructions for processor 202 to compare the images taken during the current procedure with images from previous procedures. In some cases, these previous images include a topographic representation of an area of the patient, such as the GI tract or other selected area. Software application 208 may have further sets of instructions for processor 202 to determine, for example, if the physician has examined the entire area selected for examination (e.g., by comparing the current images with previous images that represent the entire area). The processor 202 may make this determination in real-time to alert the physician that, for example, the examination has not been completed. In other embodiments, software application(s) 208 may have sets of instructions for the processor 202 to save the images in memory 212 so that the physician can confirm that the examination has been complete.

In other embodiments, the previous images may include selected tissue or areas from the patient, such as a medical disorder. The medical disorder may, for example, include a tumor, polyp, ulcer, inflammation, diseased tissue or other disorder. In this embodiment, software application(s) 208 include sets of instructions for the processor 202 to compare the current images of the disorder with previous images in memory 212 to, for example, allow the medical practitioner to determine if the disorder has changed between the procedures. For example, processor 202 may determine if a cancerous tissue has grown or changed in any material aspect. In another example, processor 202 may determine if a previously-removed polyp or cancerous tissue has returned or was completely removed in a previous procedure.

In other embodiments, memory 212 contains images and/or data of representative tissue from patients other than the current patient. For example, the representative tissue may comprise a series of images of certain types of disorders, such as a tumor, polyp, ulcer, lesion, inflammation or a cancerous or otherwise diseased tissue. In this embodiment, software application(a) 108 include a set of instructions for processor 202 to recognize and diagnose the disorder in the patient based on the images captured by imaging device 204 and the images of the representative tissue. Processor 202 may include an artificial neural network (e.g., an artificial intelligence or machine-learning program) that allows software application(s) 208 to “learn” from previous images and apply this learning to the images captured from the patient. Software application(s) 208 can be used to, for example, supplement the physician's diagnosis of the disorder based on the series of images of other similar disorders and/or to reduce the variation in diagnostic accuracy among medical practitioners.

In certain embodiments, software application(s) 208 may include sets of instructions for processor 202 to analyze images from the entire area of the procedure and compare these images with data or other images in memory 212. Software application(s) 208 may include further sets of instructions for processor 202 to detect a potential disorder in the selected area of examination based on the images and data within memory 212. Detection of a potential disease or disorder by software application 208 during the endoscopic diagnosis makes it possible to prevent a detection target from being overlooked by a medical practitioner, thereby increasing the confidence of an endoscopic diagnosis.

In certain embodiments, memory 212 includes a variety of different patient characteristics that create a patient profile, such as age, ethnicity, nationality, race, height, weight, baseline vitals, such as blood pressure, heart rate and the like, hematology results, blood chemistry or urinalysis results, physical examinations, medication usage, blood type, BMI index, prior medical history (e.g., diabetes, prior cancerous events, irritable bowel syndrome or other GI tract issues, frequency of colonoscopies, frequency and growth rate of polyps, etc.) and other relevant variables. Memory 212 may be linked to a central repository in a computer network or similar type network that provides similar profiles from a multitude of different patients in different locations around the country. In this manner, an individual health care practitioner or hospital staff can access hundreds or thousands of different patient profiles from various locations around the country or the world.

In this embodiment, software application 208 may include an artificial neural network capable of classifying the patient based on a comparison of his/her individual profile and the other profiles in the network. This classification may include a relevant risk profile for the patient to develop certain disorders or diseases. Alternatively, it may allow the software application 208 to diagnose the patient based on the images and/or data collected during the procedure.

In another embodiment, software application 208 and memory 212 are configured to maintain records of a particular health care provider (e.g., endoscopist) and/or health center (e.g., hospital, ASC or the like) related to the procedures performed by that health care provider or health center. These records may, for example, include the number of colonoscopies performed by a health care provider, the results of such procedures (e.g., detection of a disorder, time spent for the procedure and the like). Software application 208 is configured to capture the data within memory 212 and compute certain attributes for each particular health care provider or health center. For example, software application 208 may determine a disorder detection rate of a particular health care provider and compare that rate versus other health care providers or health centers.

Certain institutions, such as health insurance companies, may be particularly interested in comparing such data across different health care providers or health centers. For example, software application 208 may be configured to measure the adenoma detection rate of a particular health care provider or health center and compare that rate to other health care providers or to an overall average that has been computed from the data in memory 212. This adenoma detection rate can, for example, be used to profile a health care provider or, for example, as a quality control for insurance purposes.

In certain embodiments, the processor and/or software applications 108 are configured to record the time throughout the procedure and to capture the exact time of certain events during the procedure, such as the start time (i.e., the time the endoscope is advanced into the patient's body), the time that the endoscope captures images of certain disorders or certain target areas within the patient, the withdrawal time and the like. Software application 208 is configured to measure, for example, the time spent for the entire procedure, the time spent from entry into the patient to image capture of a certain disorder and the like. This data can be collected into memory 212 for later use. For example, an insurance provider may desire to know the amount of time a surgeon spends in a procedure or the amount of time it takes from entry into the patient until the surgeon reaches a particular disorder, such as a lesion, tumor, polyp or the like.

Data gathered from any of the sources above may be used to train an algorithm, such as an AI algorithm, to predict exacerbations or flare-ups. Information input regarding medication may be used to, for example, predict or otherwise consider a patient's response to medication and enable a health care provider, patient, caregiver or other party to tailor medication treatments. Data from different sources described above may be combined in various permutations in order to enable predictive diagnostics and/or treatment recommendations.

The artificial neural network within processor 202 may be configured to perform a difference analysis between the images captured by imaging device 204 and a prediction image. The prediction image may be generated based on images of representative tissue within memory 212 or other tissue data that has been downloaded onto processor 202. The difference analysis may include, but is not limited to, comparing textures, colors, sizes, shapes, spectral variations, biomarkers, or other characteristics of the images captures by imaging device 204 and the prediction image.

In certain embodiments, diagnostic system 200 is part of a larger network that may include hundreds or thousands of other systems similar to system 200. In this embodiment, when system 200 recognizes a medical condition or disorder and provides a preliminary diagnosis of that condition or disorder, this information may be communicated back to a central processor or computer server (not shown) that is managed as part of a proprietary system. This information may be accumulated from multiple independent users of the system located in remote locations (i.e., different hospitals around the country). The accumulated data may be examined for quality control and then added to a larger database. This added data may be used to further calibrate and fine-tune the overall system for improved performance. The artificial neural network continually updates memory 212 and software application(s) 208 to improve the accurate of diagnosis of these disorders.

In addition, the artificial neural network in processor 202 may be configured to generate a confidence value for the diagnosis of a particular disorder or disease. The confidence level may, for example, illustrate a level of confidence that the disease is present in the tissue based on the images taken thereof. The confidence value(s) may also be used, for example, to illustrate overlapping disease states and/or margins of the disease type for heterogenous diseases and the level of confidence associated with the overlapping disease states.

In certain embodiments, the artificial neural network in processor 202 may include sets of instructions to grade certain diseases, such as cancer. The grade may, for example, provide a degree of development of the cancer from an early stage of development to a well-developed cancer (e.g., Grade 1, Grade 2, etc.). In this embodiment, software application(s) 208 include a set of instructions for processor 202 to compare the characteristics of an image captured by imaging device 204 with data from memory 212 to provide such grading.

In addition, system 200 may include a set of instructions for processor 202 to distinguish various disease types and sub-types from normal tissue (e.g., tissue presumed to have no relevant disease). In this embodiment, system 200 may differentiate normal tissue proximal to a cancerous lesion and normal tissue at a distal location from the cancerous lesion. The artificial neural network may be configured to analyze the proximal normal tissue, distal normal tissue and benign normal tissue. Normal tissue within a tumor may have a different signature than benign lesions and proximal normal tissue may have a different signature than distal normal tissue. For example, the signature of the proximal normal tissue may indicate emerging cancer, while the signature in the distal normal tissue may indicate a different disease state. In this embodiment, system 200 may use the proximity of the tissue to the cancerous tissue to, for example, measure a relevant strength of a disease, growth of a disease and patterns of a disease.

Sensor(s) 220 are preferably disposed on, or within, one or more of the imaging devices 204 and/or the support devices 230. In certain embodiments, sensors 220 are located on a distal end portion of an endoscope (discussed below). In other embodiments, sensors 120 are located on, or within, a support device 230 attached to the distal end portion of the endoscope.

Sensor(s) 220 are configured to detect one or more physiological parameter(s) of tissue around the outer surface of the main body. The physiological parameter(s) may include a temperature of the tissue, a type of fluid in, or around, the tissue, pathogens in, or around, the tissue, a dimension of the tissue, a depth of the tissue, a tissue disorder, such as a lesion, tumor, ulcer, polyp or other abnormality, biological receptors in, or around, the tissue, tissue biomarkers, tissue bioimpedance, a PH of fluid in, or around the tissue or the like.

In certain embodiments, the sensor(s) 220 detect temperature of the tissue and transmit this temperature data to the processor. Software applications 208 include a set of instructions to compare the tissue temperature with data in memory 212 related to standard tissue temperature ranges. Processor is then able to determine if the tissue includes certain disorders based on the tissue temperature (e.g., thermography). For example, certain tumors are more vascularized than ordinary tissue and therefore have higher temperatures. The memory 212 includes temperature ranges that indicate “normal tissue” versus highly vascularized tissue. The processor can determine if the tissue is highly vascularized based on the collected temperature to indicate that the tissue may be cancerous.

In certain embodiments, sensor(s) 220 may include certain components configured to measure the topography of the tissue near the surface of the coupler device. For example, sensor(s) 220 may be capable of providing a 3-D representation of the target tissue. In certain embodiments, sensor(s) 220 are capable of measuring reflected light and capturing information about the reflected light, such as the return time and/or wavelengths to determine distances between the sensor(s) 220 and the target tissue. This information may be collected by software application 208 to create a digital 3-D representation of the target tissue.

In one embodiment, support device 230 or the endoscope further includes a light imaging device that uses ultraviolet, visible and/or near infrared light to image objects. The light may be concentrated into a narrow beam to provides very high resolutions. The light may be transmitted with a laser, such as a YAG laser, holmium laser and the like. In one preferred embodiment, the laser comprises a disposable or single-use laser fiber mounted on or within the optical coupler device. Alternatively, the laser may be advanced through the working channel of the endoscope and the optical coupler device.

Sensor(s) 220 are capable of receiving and measuring the reflected light from the laser (e.g., LIDAR or LADAR) and transmitting this information to the processor. In this embodiment, one or more software applications 208 are configured to transform this data into a 3-D map of the patient's tissue. This 3-D map may can be used to assist with the diagnosis and/or treatment of disorders in the patient.

In another embodiment, monitoring system 200 includes an ultrasound transducer, probe or other device configured to produce sound waves and bounce the sound waves off tissue within the patient. The ultrasound transducer receives the echoes from the sound waves and transmits these echoes to the processor. The processor includes one or more software applications 208 with a set of instructions to determine tissue depth based on the echoes and/or produce a sonogram representing the surface of the tissue. The ultrasound probe may be delivered through a working channel in the endoscope and the optical coupler device. Alternatively, the transducer may be integrated into either the endoscope or the support device. In this latter embodiment, the transducer may be, for example, a disposable transducer within the support device that receives electric signals wirelessly, or through a connector extending through the endoscope.

Suitable sensors 220 for use with the present invention may include PCT and microarray based sensors, optical sensors (e.g., bioluminescence and fluorescence), piezoelectric, potentiometric, amperometric, conductometric, nanosensors or the like. Physical properties that can be sensed include temperature, pressure, vibration, sound level, light intensity, load or weight, flow rate of gases and liquids, amplitude of magnetic and electronic fields, and concentrations of many substances in gaseous, liquid, or solid form. Sensors 220 can measure anatomy and movement in three dimensions using miniaturized sensors, which can collect spatial data for the accurate reconstruction of the topography of tissue in the heart, blood vessels, gastrointestinal tract, stomach, and other organs. Pathogens can also be detected by another biosensor, which uses integrated optics, immunoassay techniques, and surface chemistry. Changes in a laser light transmitted by the sensor indicate the presence of specific bacteria, and this information can be available in hours

Sensors 220 can measure a wide variety of parameters regarding activity of the selected areas in the patient, such as the esophagus, stomach, duodenum, small intestine, and/or colon. Depending on the parameter measured, different types of sensors 220 may be used. For example, sensor 220 may be configured to measure pH via, for example, chemical pH sensors. Gastric myoelectrical activity may be measured via, for example, electrogastrography (“EGG”). Gastric motility and/or dysmotility may be measured, via, for example, accelerometers, gyroscopes, pressure sensors, impedance gastric motility (IGM) using bioimpedance, strain gauges, optical sensors, acoustical sensors/microphones, manometry, and percussive gastogram. Gut pressure and/or sounds may be measured using, for example, accelerometers and acoustic sensors/microphones.

Sensors 220 may include acoustic, pressure, and/or other types of sensors to identify the presence of high electrical activity but low muscle response indicative of electro-mechanical uncoupling. When electro-mechanical uncoupling occurs, sensors 220, alone or in combination with the other components of monitoring system 200, may measure propagation of slow waves in regions such as the stomach, intestine, and colon.

In certain embodiments, system 200 may be configured to capture data relevant to actual size and depth of tissue, lesions, ulcers, polyps, tumors and/or other abnormalities within the patient. For example, the size of a lesion or ulcer may range from a scale of 100 micrometers to a few centimeters. Software applications 208 may be configured to collect this depth information and to classify the depth as being superficial, submucosal, and/or muscularis. System 200 also be configured to capture data regarding the prevalence of impact of lesions or ulcers within a specific region of the patient.

Data gathered from any of the sources above may be used to train an algorithm, such as an AI algorithm, to predict exacerbations or flare-ups. Information input regarding medication may be used to, for example, predict or otherwise consider a patient's response to medication and enable a health care provider, patient, caregiver or other party to tailor medication treatments. Data from different sources described above may be combined in various permutations in order to enable predictive diagnostics and/or treatment recommendations.

System 200 may further be configured to capture information regarding inflammation. For example, imaging device 204 may be capable of capturing data regarding vasculature including patchy obliteration and/or complete obliteration, dilation or over-perfusion, data related to perfusion information and real-time perfusion information, data relevant to blood's permeation into a tissue or data relevant to tissue thickening, which may be the result of increased blood flow to a tissue and possible obliteration of blood vessels and/or inflammation. Software applications 208 are configured to process this data and compare it to information or data within memory 212 to provide a more accurate diagnosis to the physician.

System 200 may also be configured to measure stenosis in a target lumen within the patient, such as the GI tract, by assessing the amount of narrowing in various regions of the target lumen. System 200 may also be configured to assess, for example, tissue properties such as stiffness. For example, stiffness may be monitored during expansion of a balloon or stent to prevent unwanted fissures or damage.

Imaging device 204 may further be configured to assess bleeding. For example, imaging device 204 may capture data relevant to spots of coagulated blood on a surface of mucosa which can implicate, for example, scarring. Imaging device 204 may also be configured to capture data regarding free liquid in a lumen of the GI tract. Such free liquid may be associated with plasma in blood. Furthermore, imaging device 204 may be configured to capture data relevant to hemorrhagic mucosa and/or obliteration of blood vessels.

Software application 208 may further be configured to process information regarding lesions, ulcers, tumors and/or other tissue abnormalities. For example, software application 208 may also be configured to accurately identify and assess the impact of lesions and/or ulcers on one or more specific regions of the GI tract. For example, software application 208 may compare the relative prevalence of lesions and/or ulcers across different regions of the GI tract. For example, software application 208 may calculate the percentage of affected surface area of a GI tract and compare different regions of the GI tract. As a further example, software application 208 may quantify the number of ulcers and/or lesions in a particular area of the GI tract and compare that number with other areas of the GI tract. Software application 208 may also consider relative severity of ulcers and/or lesions in an area of the GI tract by, for example, classifying one or more ulcers and/or lesions into a particular pre-determined classification, by assigning a point scoring system to ulcers and/or lesions based on severity, or by any other suitable method.

Software application 208, along with one or more imaging devices 204 and/or support devices 230, may be configured to quantify severity of one or more symptoms or characteristics of a disease state. For example, software application 208 may be configured to assign quantitative or otherwise objective measure to one or more disease conditions such as ulcers/lesions, tumors, inflammation, stenosis, and/or bleeding. Software application 208 may also be configured to assign a quantitative or otherwise objective measure to a severity of a disease as a whole. Such quantitative or otherwise objective measures may, for example, be compared to one or more threshold values in order to assess the severity of a disease state. Such quantitative or otherwise objective measures may also be used to take preventative or remedial measures by, for example, administering treatment through a therapy delivery system as discussed below or by providing an alert (e.g., to medical personnel, a patient, or a caregiver).

Software application 208 may store the results or any component of its analyses, such as quantitative or otherwise objective measures, in memory 212. Results or information stored in memory 212 may later be utilized for, for example, tracking disease progression over time. Such results may be used to, for example, predict flare-ups and take preventative or remedial measures by, for example, administering treatment through a therapy delivery system as discussed or by providing an alert (e.g., to medical personnel, a patient, or a caregiver).

Imaging device 204 and/or support device 230 may be in communication either directly or indirectly with software application 208, which may be stored on a processor or other suitable hardware. Imaging device 204 may be connected with software application 208 by a wired or wireless connection. Alternatively, imaging device 204 may be in communication with another type of processing unit. Software application 208 may run on a specialized device, a general-use smart phone or other portable device, and/or a personal computer. Software application 208 may also be part of an endoscope system, endoscope tool, wireless endoscopic capsule, or implantable device which also includes imaging device 204. Software application 208 may be connected by a wired or wireless connection to imaging device 204, memory 212, therapy delivery system 216 and/or sensors 220.

Imaging device 204 may be configured to capture images at one or more locations at target site(s) within the patient. Imaging device 204, a device carrying imaging device 204, or another component of monitoring system 2100, such as software application 208, may be capable of determining the location of the target site where images were recorded. Imaging device 204 may capture images continually or periodically.

Imaging device 204 may be any imaging device capable of taking images including optical, infrared, thermal, or other images. Imaging device 204 may be capable of taking still images, video images, or both still and video images. Imaging device 204 may be configured to transmit images to a receiving device, either through a wired or a wireless connection. Imaging device 204 may be, for example, a component of an endoscope system, a component of a tool deployed in a working port of an endoscope, a wireless endoscopic capsule, or one or more implantable monitors or other devices. In the case of an implantable monitor, such an implantable monitor may be permanently or temporarily implanted.

In certain embodiments, imaging device 104 is an endoscope. The term “endoscope” in the present disclosure refers generally to any scope used on or in a medical application, which includes a body (human or otherwise) and includes, for example, a laparoscope, duodenoscope, endoscopic ultrasound scope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure. For example, visualization inside the gastrointestinal tract may involve the use of endoscopy in the form of flexible gastroscopes and colonoscopes, endoscopic ultrasound scopes (EUS) and specialty duodenum scopes with lengths that can run many feet and diameters that can exceed 1 centimeter. These scopes can be turned and articulated or steered by the physician as the scope is navigated through the patient. Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope), chest and thoracic cavity (thoracoscope), and the heart (cardioscope). In addition, robotic medical devices rely on scopes for remote visualization of the areas the robotic device is assessing and treating.

These and other scopes may be inserted through natural orifices (such as the mouth, sinus, ear, urethra, anus and vagina) and through incisions and port-based openings in the patient's skin, cavity, skull, joint, or other medically indicated points of entry. Examples of the diagnostic use of endoscopy with visualization using these medical scopes includes investigating the symptoms of disease, such as maladies of the digestive system (for example, nausea, vomiting, abdominal pain, gastrointestinal bleeding), or confirming a diagnosis, (for example by performing a biopsy for anemia, bleeding, inflammation, and cancer) or surgical treatment of the disease (such as removal of a ruptured appendix or cautery of an endogastric bleed).

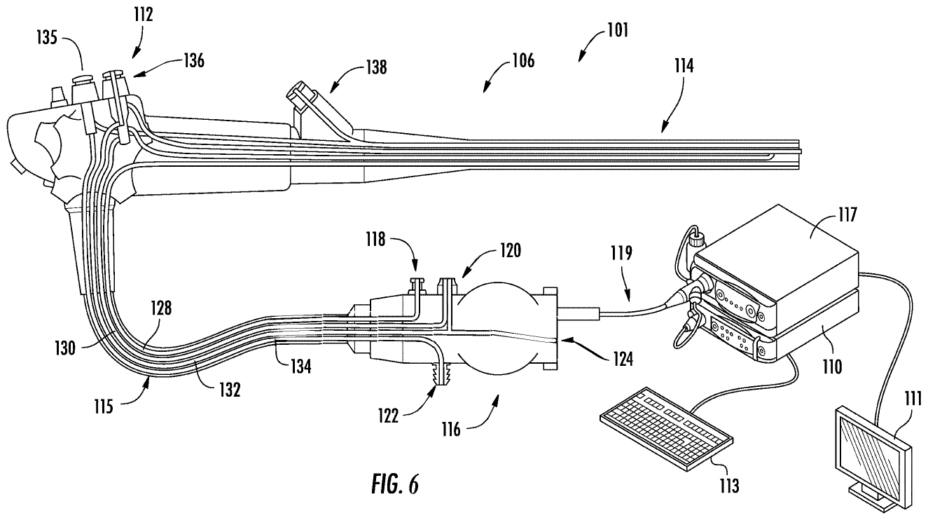

As illustrated in FIG. 6, a representative endoscope system 101 has an endoscope 106, a light source device 117, a processor 110, a monitor 111 (display unit), and a console 113. The endoscope 106 is optically connected to the light source device 117 and is electrically connected to the processor device 110. The processor device 110 is electrically connected to the monitor 111 and the console 113. The monitor 111 outputs and displays an image of an observation target, information accompanying the image, and so forth. The console 113 functions as a user interface that receives an input operation of designating a region of interest, setting a function, or the like.

The illumination light emitted by the light source unit 117 passes through a light path coupling unit 119 formed of a mirror, a lens, and the like and then enters a light guide built in the endoscope 106 and a universal cord 115, and causes the illumination light to propagate to the distal end portion 114 of the endoscope 106. The universal cord 115 is a cord that connects the endoscope 106 to the light source device 117 and the processor device 110. A multimode fiber may be used as the light guide.

The hardware structure of a processor 110 executes various processing operations, such as the image processing unit, and may include a central processing unit (CPU), which is a general-purpose processor executing software (program) and functioning as various processing units; a programmable logic device (PLD), which is a processor whose circuit configuration is changeable after manufacturing, such as a field programmable gate array (FPGA); a dedicated electric circuit, which is a processor having a circuit configuration designed exclusively for executing various processing operations, and the like.

FIG. 6 also illustrates a representative endoscope 106 for use with the present disclosure including a proximal handle 127 adapted for manipulation by the surgeon or clinician coupled to an elongate shaft 114 adapted for insertion through a natural orifice or an endoscopic or percutaneous penetration into a body cavity of a patient. Endoscope 100 further includes a fluid delivery system 125 coupled to handle 127 via a universal cord 115. Fluid delivery system 125 may include a number of different tubes coupled to internal lumens within shaft 114 for delivery of fluid(s), such as water and air, suction, and other features that may be desired by the clinician to displace fluid, blood, debris and particulate matter from the field of view. This provides a better view of the underlying tissue or matter for assessment and therapy. In the representative embodiment, fluid delivery system 125 includes a water-jet connector 118, water bottle connector 121, a suction connector 122 and an air pipe 124. Water jet connector 118, water bottle connector 121, suction connector 122 and air pipe 124 are each connected to internal lumens 128, 130, 132, 134 respectively, that pass through shaft 114 to the distal end of endoscope 100.

Endoscope 100 may further include a working channel (not shown) for passing instruments therethrough. The working channel permits passage of instruments down the shaft 114 of endoscope 100 for assessment and treatment of tissue and other matter. Such instruments may include cannula, catheters, stents and stent delivery systems, papillotomes, wires, other imaging devices including mini-scopes, baskets, snares and other devices for use with a scope in a lumen.

Proximal handle 127 may include a variety of controls for the surgeon or clinician to operate fluid delivery system 125. In the representative embodiment, handle 127 include a suction valve 135, and air/water valve 136 and a biopsy valve 138 for extracting tissue samples from the patient. Handle 127 will also include an eyepiece (not shown) coupled to an image capture device (not shown), such as a lens and a light transmitting system. The term “image capture device” as used herein also need not refer to devices that only have lenses or other light directing structure. Instead, for example, the image capture device could be any device that can capture and relay an image, including (i) relay lenses between the objective lens at the distal end of the scope and an eyepiece, (ii) fiber optics, (iii) charge coupled devices (CCD), (iv) complementary metal oxide semiconductor (CMOS) sensors. An image capture device may also be merely a chip for sensing light and generating electrical signals for communication corresponding to the sensed light or other technology for transmitting an image. The image capture device may have a viewing end—where the light is captured. Generally, the image capture device can be any device that can view objects, capture images and/or capture video.

In some embodiments, endoscope 100 includes some form of positioning assembly (e.g., hand controls) attached to a proximal end of the shaft to allow the operator to steer the scope. In other embodiments, the scope is part of a robotic element that provides for steerability and positioning of the scope relative to the desired point to investigate and focus the scope.

In certain embodiments, imaging device 204 is an endoscope. The term “endoscope” in the present disclosure refers generally to any scope used on or in a medical application, which includes a body (human or otherwise) and includes, for example, a laparoscope, duodenoscope, endoscopic ultrasound scope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure. For example, visualization inside the gastrointestinal tract may involve the use of endoscopy in the form of flexible gastroscopes and colonoscopes, endoscopic ultrasound scopes (EUS) and specialty duodenum scopes with lengths that can run many feet and diameters that can exceed 1 centimeter. These scopes can be turned and articulated or steered by the physician as the scope is navigated through the patient. Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope), chest and thoracic cavity (thoracoscope), and the heart (cardioscope). In addition, robotic medical devices rely on scopes for remote visualization of the areas the robotic device is assessing and treating.

These and other scopes may be inserted through natural orifices (such as the mouth, sinus, ear, urethra, anus and vagina) and through incisions and port-based openings in the patient's skin, cavity, skull, joint, or other medically indicated points of entry. Examples of the diagnostic use of endoscopy with visualization using these medical scopes includes investigating the symptoms of disease, such as maladies of the digestive system (for example, nausea, vomiting, abdominal pain, gastrointestinal bleeding), or confirming a diagnosis, (for example by performing a biopsy for anemia, bleeding, inflammation, and cancer) or surgical treatment of the disease (such as removal of a ruptured appendix or cautery of an endogastric bleed).

In certain embodiments, support device 10 may include (or a a fluid sample lumen (not shown) configured to withdraw tissue and/or fluid samples from the patient for analysis. The fluid sample lumen may have a proximal end coupled to a fluid delivery system (not shown) for delivering a fluid, such as water, through device 10 to a target site on the patient's tissue. In certain embodiments, fluid delivery system is configured to delivery one or more droplets of water through device 10.

In certain embodiments, the fluid sample lumen may also have a proximal end coupled to a gas delivery system configured to deliver a gas through device 10 such that the gas interacts with the fluid droplets and the tissue or fluid sample from the patient. In a preferred embodiment, the fluid droplets and the gas are delivered to device 10 so as to collect small molecules from the tissue or fluid sample of the patient. These small molecules are then withdrawn from the patient.

The fluid or tissue sample withdrawn through the fluid sample lumen may be analyzed by a variety of different tissue analyzing devices known in the art, such as a mass spectrometer, cold vapor atomic absorption or fluorescence devices, histopathologic devices and the like. In a preferred embodiment, the tissue analyzing device includes a particle detector, such as mass analyzer or mass spectrometer, coupled to the ionizer and configured to sort the ions, preferably based on a mass-to-charge ratio, and a detector coupled to the mass analyzer and configured to measure a quantity of each of the ions after they have been sorted. Monitoring system 100 further comprises one or more software application(s) coupled to the detector and configured to characterize a medical condition of the patient based on the quantity of each of the ions in the tissue sample. The medical condition may include a variety of disorders, such as tumors, polyps, ulcers, diseased tissue, pathogens or the like. In one embodiment, the medical condition comprises a tumor and the processor is configured to diagnose the tumor based on the quantity of each of the ions retrieved from the tissue sample. For example, the processor may be configured to determine the type of proteins or peptides existing in a tissue sample based on the type and quantity of ions. Certain proteins or peptides may provide information to the processor that the tissue sample is, for example, cancerous or pre-cancerous.

The particle detector, such as a mass spectrometer, may be coupled to device 10 to analyze the tissue or fluid sample withdrawn from the patient. In certain embodiments, the particle detector further comprises a heating device configured to vaporize the tissue sample and an ionization source, such as an electron beam or other suitable ionizing device to ionize the vaporized tissue sample by giving the molecules in the tissue sample a positive electric charge (i.e., either by removing an electron or adding a proton). Alternatively, the heating device and/or electron beam may be incorporated directly into support device 10 or scope 230 so that the tissue sample is vaporized and/or ionized before it is withdrawn from scope 230.

The particle detector may further include a mass analyzer for separating the ionized fragments of the tissue sample according to their masses. In one embodiment, the mass analyzer comprises a particle accelerator and a magnet configured to create a magnetic field sufficient to separate the accelerated particles based on their mass/charge ratios. The particle detector further comprises a detector at a distal end of particle detector for detecting and transmitting data regarding the various particles from the tissue sample.

According to the present disclosure, a software application 108, such as the machine-learning or artificial intelligent software application described above, may be coupled to the particle detector to analyze the detected particles. For example, the software application may determine the type of proteins or peptides within the tissue sample based on their mass-to-charge ratios. The software application may further determine, based on data within memory 112, whether the proteins or peptides indicate cancerous tissue in the patient. Alternatively, software application 108 may determine molecular lesions such as genetic mutations and epigenetic changes that can lead cells to progress into a cytologically preneoplastic or premalignant form.

Hereby, all issued patents, published patent applications, and non-patent publications that are mentioned in this specification are herein incorporated by reference in their entirety for all purposes, to the same extent as if each individual issued patent, published patent application, or non-patent publication were specifically and individually indicated to be incorporated by reference.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. A support device for an endoscope having a distal end, the support device comprising:
a tubular member configured for removable attachment to an outer surface of the endoscope near, or at, the distal end, the tubular member having an outer surface;
a plurality of projecting elements extending outward from the outer surface of the tubular member, the projecting elements being spaced from each other around a circumference of the tubular member; and
an optically transparent cover coupled to the tubular member and configured for covering the distal end of the endoscope when the tubular member is attached to the outer surface of the endoscope.

2. The support device of claim 1, wherein the cover is spaced from the distal end of the endoscope when the tubular member is attached to the outer surface of the endoscope.

3. The support device of claim 2, wherein the endoscope has a lens at the distal end of the endoscope, and wherein the cover is spaced from the lens by a length less than a minimum focal distance of the endoscope.

4. The support device of claim 1, wherein the cover and the tubular member create a seal over the distal end of the endoscope.

5. The support device of claim 4, wherein the cover is integral with the tubular member to form a unitary body.

6. The support device of claim 1, wherein the tubular member has an inner surface configured for gripping the outer surface of the endoscope.

7. The support device of claim 1, wherein each of the projecting elements comprise a base coupled to the tubular member and a substantially flexible arm extending from the base.

8. The support device of claim 1, wherein the flexible arm of each projecting element is movable between a first position, wherein the flexible arm is substantially perpendicular to a longitudinal axis of the tubular member, to a second position, wherein the flexible arm extends transversely to the longitudinal axis of the tubular member.

9. The support device of claim 8, wherein the flexible arms extend substantially perpendicular to the longitudinal axis of the tubular member in the second position.

10. The support device of claim 1, further comprising about 2 to about 20 projecting elements.

11. The support device of claim 1, further comprising one or more sensors on the tubular member or the cover for detecting a physiological parameter of a patient.

12. A support device for an endoscope having a distal end, the support device comprising:
a tubular member configured for removable attachment to an outer surface of the endoscope near, or at, the distal end, the tubular member having an outer surface; and
first and second rings of projecting elements extending outward from the outer surface of the tubular member, the projecting elements within the first and second rings being spaced from each other around a circumference of the tubular member to define gaps therebetween, wherein the first ring is spaced longitudinally from the second ring and wherein the projecting elements of the second ring are aligned longitudinally with the gaps between the projecting elements in the first ring.

13. The support device of claim 12, wherein the projecting elements of the first ring are aligned longitudinally with the gaps between the projecting elements in the second ring.

14. The support device of claim 12, wherein the second ring is spaced from the first ring by a distance of greater than about 2.5 cm.

15. The support device of claim 12, further comprising an optically transparent cover coupled to the tubular member and configured for covering the distal end of the endoscope when the tubular member is attached to the outer surface of the endoscope.

16. The support device of claim 15, wherein the cover is spaced from the distal end of the endoscope when the tubular member is attached to the outer surface of the endoscope.

17. The support device of claim 16, wherein the endoscope has a lens at the distal end of the endoscope, and wherein the cover is spaced from the lens by a length less than a minimum focal distance of the endoscope.

18. The support device of claim 12, wherein the tubular member has an inner surface configured for gripping the outer surface of the endoscope.

19. The support device of claim 12, wherein each of the projecting elements comprise a base coupled to the tubular member and a substantially flexible arm extending from the base.

20. The support device of claim 19, wherein the flexible arm of each projecting element is movable between a first position, wherein the flexible arm is substantially perpendicular to a longitudinal axis of the tubular member, to a second position, wherein the flexible arm extends transversely to the longitudinal axis of the tubular member.

21. The support device of claim 20, wherein the flexible arms extends substantially parallel to the longitudinal axis of the tubular member in the second position.

22. The support device of claim 12, further comprising about 2 to about 20 projecting elements.

23. The support device of claim 12, further comprising one or more sensors on the tubular member for detecting a physiological parameter in a patient.

24. A support device for an endoscope having a distal end, the support device comprising:
a tubular member configured for removable attachment to an outer surface of the endoscope near, or at, the distal end, the tubular member having an outer surface; and
a plurality of projecting elements extending outward from the outer surface of the tubular member, the projecting elements being spaced from each other around a circumference of the tubular member; and
wherein the projecting members are spaced from a distal end of the tubular member by a distance of greater than about 20 mm.

25. The support device of claim 24, further comprising a plurality of rings of the projecting elements extending outward from the outer surface of the tubular member, wherein a distalmost ring is spaced from the distal end of the tubular member by a distance of greater than about 20 mm.

26. The support device of claim 25, wherein the projecting elements within each of the rings is offset from the projecting elements in an adjacent ring.

27. The support device of claim 25, wherein each ring of projecting members is spaced from adjacent rings by a distance of greater than about 2.5 cm.

28. The support device of claim 24, further comprising an optically transparent cover coupled to the tubular member and configured for covering the distal end of the endoscope when the tubular member is attached to the outer surface of the endoscope.

29. The support device of claim 28, wherein the cover is spaced from the distal end of the endoscope when the tubular member is attached to the outer surface of the endoscope.

30. The support device of claim 28, wherein the endoscope has a lens at the distal end of the endoscope, and wherein the cover is spaced from the lens by a length less than a minimum focal distance of the endoscope.
